(19) United States
(12) Patent Application Publication
(10) Pub. No.: US 2023/0099068 A1
(43) Pub. Date: Mar. 30, 2023

(54) TOPICAL ANTIMICROBIAL FORMULATIONS

(71) Applicant: Michael Purnell, Modesto, CA (US)

(72) Inventor: Michael Purnell, Modesto, CA (US)

(21) Appl. No.: 17/955,543

(22) Filed: Sep. 29, 2022

Related U.S. Application Data
(60) Provisional application No. 63/249,577, filed on Sep. 29, 2021.

(51) Int. Cl. A61K 31/05; A61K 9/00; A61K 31/045; A61K 31/015; A61K 47/02; A61K 47/12; A61K 47/10; A61K 47/44; A61K 47/36; A61K 47/14

(52) U.S. Cl. CPC A61K 31/05; A61K 9/0014; A61K 31/045; A61K 31/015; A61K 47/02; A61K 47/12; A61K 47/10; A61K 47/44; A61K 47/36; A61K 47/14

(57) ABSTRACT

The present formulation in one embodiment is a cream that utilizes cannabinoids and terpenes (essential oils) which have powerful antibacterial activity, proven through multiple rounds of in vitro testing, to kill the most prevalent indigenous skin bacteria, including Streptococcus, Staphylococcus, and P. acnes within 30 seconds of contact. There is some independent antimicrobial activity of the cream base as well. The cream is more suitable to use on human skin because it is an emollient, is soothing, smells and feels good, and it penetrates below the dermis (lower skin level) to enter the sebaceous glands where P. acnes bacteria resides.

This application claims priority to U.S. Provisional Patent Application 63/249,577 filed on Sep. 29, 2021, which is incorporated by reference herein in its entirety.

### BACKGROUND OF THE SYSTEM

There are a number of situations that can lead to infection in a person. Surgical site infections are often the result of contamination from indigent skin bacteria. Some people are hosts, or carriers of more bacteria than others. Particularly difficult infections to treat include MRSA (methicillin resistant Staph aureus), difficult to identify and treat is Propionibacterium acnes, or Cutibacterium acnes bacteria (aka “P. acnes”). This is the source of acne, and also of many surgical infections (e.g., around the shoulder). To date, standard preventative measures have not been effective or reliable in significantly reducing these serious surgical and other infections. The cost associated with the treatment of such infections is enormous, and the loss of quality of life also very significant.

In the prior art, the medical industry has adopted the use of 2% chlorhexidine gluconate cloth wipes and 2% topical solutions prior to surgery in order to reduce the number of host bacteria living on human skin. Despite the surgical industry's attempt to minimize surgical site infections, they still occur, with devastating consequences. The treatment of acne includes many topical and oral medications. The skin wipes have not conclusively reduced surgical site infections derived from skin bacteria in controlled studies. At best, there are retrospective studies that show some efficacy, reported by the company selling these skin wipes (e.g., Sage) as reducing hip, knee, colorectal, and Caesarian section infections between 60-73%. The wipes are known to cause allergic reactions, serious skin rashes, pain, and secondary infections, that have even led to deaths. Acne treatments have many side effects, including local toxicity as well as systemic illnesses.

### SUMMARY

The present formulation in one embodiment is a cream that utilizes cannabinoids and terpenes (essential oils) which have powerful antibacterial activity, proven through multiple rounds of in vitro testing, to kill the most prevalent indigenous skin bacteria, including Streptococcus, Staphylococcus, and P. acnes within 30 seconds of contact. There is some independent antimicrobial activity of the cream base as well. The cream is more suitable to use on human skin because it is an emollient, is soothing, smells and feels good, and it penetrates below the dermis (lower skin level) to enter the sebaceous glands where P. acnes bacteria resides.

The endocannabinoid system also has receptors at the subdermal level, and when cannabinoids bind to these, they can provide an anti-inflammatory effect and act as a pain reliever. This formulation does not have any of the side effects or complications of the chlorhexidine wipes or acne treatment regimens for most non-hypersensitive users. Use of natural plant extracts and derivatives is also very appealing to many people, compared to manufactured drugs that have been artificially manipulated.

### DETAILED DESCRIPTION OF THE SYSTEM

The present formulation is a proprietary formula that possesses unique antimicrobial and anti-inflammation properties. Derivatives of the formulation are also contemplated to develop soaps, lotions, and other topically applied materials with antibacterial and antiviral properties. These may be relevant to prevent and treat other skin infections in addition to acne and surgical site infections. In one embodiment, the formulation can be used as part of a pre-operative protocol to reduce surgery related infections. The formulation can also be used topically to help prevent acne breakouts, to act as an anti-inflammatory, and to help fight existing infections.

In one embodiment, the formulation comprises a cannabinoid for the treatment or prevention of infection via topical application. The cannabinoid is chosen from the list comprising cannabidiol, cannibiol, cannabigeraol, cannabi-chromene, and tetrahydrocannabinol. The composition comprises a cannabinoid at a concentration of between 0.1 mg/ml and 10 mg/ml.

The formulation is such as to deliver a therapeutically effective amount of the cannabinoid to the dermal or mucosal surface of the subject. Therapeutically effective amount refers to an amount sufficient to inhibit bacterial growth associated with bacterial carriage and/or bacterial infection of the skin.

In one embodiment, the formulation includes cannabinoid and one or more terpenes resulting in enhanced bioavailability and enhanced solubility of CBD for treating p acnes and/or other infections.

In one embodiment, the formulation includes a beeswax in a concentration range of 2 mg/ml to 20 mg/ml. In one embodiment, the formulation includes a beeswax in a concentration of 5 mg/ml to 10 mg/ml. In embodiment, the formulation includes a beeswax in a percentage of %5 to 9%. In one embodiment, the beeswax may be PEG8 Beeswax.

In one embodiment, the formulation includes Tefose HC, Polipid 141, and Cithrol GMS 40 in various combinations. The formulation avoids salicylic acid, mineral salts (zinc, copper, silver, and the like). In one embodiment the formulation uses one or more terpenes in various combinations from a list including, but not limited to, linalool, geraniol, menthol, pinene, and the like.

The present formulation in an embodiment includes glycerin, butylated hydroxy toluene, distilled water, and the like in a water phase, and terpenes, cannabidiol, corn oil, MCT, Tefose HC, prolipid, beeswax, cithrol and carrageenan in an oil phase. The oil phase ingredients in one embodiment are heated to 75-80 degrees C. to dissolution. The water phase ingredients are combined and heated in the same temperature range until dissolution. The mixtures are combined (water phase to oil phase) while stirring until the mixture cools to less than 40 degrees C. and starts to solidify.

Table 1 illustrates embodiments of the formulation with the ingredients shown as percent of the total formulation.

Water Phase

| Ingredient | A | B | C | D | E |
|---|---|---|---|---|---|
| Zinc Sulfate | 0 | 3 | 3 | 3 | 3 |
| Salicylic Acid | 0 | 0 | 1 | 0 | 1 |
| Copper Sulfate | 0 | 0 | 0 | 3 | 3 |
| Glycerin | 5 | 5 | 5 | 5 | 5 |
| Citric Acid | 1 | 1 | 1 | 1 | 1 |
| Butylated Hydroxy | 0.05 | 0.05 | 0.05 | 005 | 0.05 |
| Toluene | | | | | |
| Distilled Water | 59.6 | 56.6 | 55.6 | 53.6 | 52.6 |

Oil Phase

| Ingredient | A | B | C | D | E |
|---|---|---|---|---|---|
| Linalool, geraniol, | 5 | 5 | 5 | 5 | 5 |
| menthol, pinene | | | | | |
| Cannabidiol | 1 | 1 | 1 | 1 | 1 |
| Corn Oil | 4 | 4 | 4 | 4 | 4 |
| MCT | 3 | 3 | 3 | 3 | 3 |
| Tefose HC | 4 | 4 | 4 | 4 | 4 |
| Prolipid 141 | 5 | 5 | 5 | 5 | 5 |
| PEG-8 Beeswax | 7 | 7 | 7 | 7 | 7 |
| Cithrol GMS 40 | 5 | 5 | 5 | 5 | 5 |
| Carrageenan | 0.35 | 0.35 | 0.35 | 0.35 | 0.35 |

The oil phase ingredients in one embodiment are heated to 75-80 degrees C. to dissolution. The water phase ingredients are combined and heated in the same temperature range until dissolution. The mixtures are combined (water phase to oil phase) while stirring until the mixture cools to less than 40 degrees C. and starts to solidify.

In one embodiment, the formulation is made with an emulsifier such as Emulium Mellifer MB, comprised of esters of jojoba wax, beeswax, and polyglycerol.

Thus, a topical antimicrobial formulation has been described.

What is claimed is:

1. A topical formulation comprising:
(a) 63 to 67% water phase formulation
(b) 33 to 37% oil phase formulation comprising antimicrobial essential oils.

2. The formulation of claim 1 wherein the water phase formulation includes one or more of zinc sulfate, salicylic acid, copper sulfate, glycerin, citric acid, butylated hydroxy toluene, and distilled water.

3. The formulation of claim 1 wherein the oil phase formulation includes one or more of linalool, geraniol, menthol, and pinene.

4. The formulation of claim 3 wherein the oil phase formulation further includes one or more of cannabidiol, corn oil, MCT, Tefose HC, Prolipd 141, PEG-8 beeswax, cithrol GMS 40, and Carrageenan.

5. The formulation of claim 1 further including Emulium Mellifer MB
